(19) United States
(12) Patent Application Publication
(10) Pub. No.: US 2014/0118518 A1
(43) Pub. Date: May 1, 2014

(54) CONTROLLED-USE IMAGING SYSTEM

(71) Applicant: Olivier Fructus, Nazelles (FR)

(72) Inventor: Olivier Fructus, Nazelles (FR)

(21) Appl. No.: 14/129,685

(22) PCT Filed: Jun. 25, 2012
(86) PCT No.: PCT/FR2012/051447

§ 371 Date: Dec. 27, 2013

(30) Foreign Application Priority Data
Jun. 28, 2011 (FR) 1155730

(51) Int. Cl. A61B 1/045

(52) U.S. Cl. CPC A61B 1/045
USPC 348/65

(57) ABSTRACT

The invention relates to an imaging system comprising:
a medical instrument (3) that is separable from a connection support (4), the separable medical instrument including an insertion tube provided with a non-volatile memory (23);
an imaging appliance (11) including a processor and image display unit connected to the viewing system; and
a control unit (20) enabling the operation of the imaging appliance (11) to be controlled and connected to a system (21) for detecting connection between the medical instrument (3) and the connection support (4). According to the invention, the non-volatile memory (23) contains identity data of the associated medical instrument and utilization data concerning the imaging appliance sent by the control unit (20) on each connection, and the control unit (20) authorizes operation of the imaging appliance (11) only if the user is identified and has given authorization. The medical instrument can thus be reused under the entire responsibility of the user.

The present invention relates to the technical field of instruments for medical purposes that make it possible to access, illuminate, and examine the inside of a body in the general sense, such as a cavity or a canal.

More precisely, the invention relates to a single-use medical endoscope suitable for performing surface or in-depth examination of a hollow organ, or of a natural or artificial cavity or duct in the human body for therapeutic, surgical, or diagnostic purposes.

For example, an endoscope of the invention may be used in the fields of urinary tracts, of gastrointestinal tracts, of the respiratory system, of the cardiovascular system, of the trachea, of the sinus cavity, of the reproductive system in women, of the abdominal cavity, or of any other part of the human body that may be explored via a natural access or an artificial access.

In the above technical field, there exist various types of endoscope that are adapted to the accessible organs that are to be observed. In general, an endoscope comprises a more or less flexible insertion tube possessing a proximal portion for connection to an actuation support that enables the tube to be steered inside the insertion passageway. The endoscope also has a generally optical viewing system such as a camera associated with a light source enabling the distal portion of the insertion tube to illuminate and examine the organ, the cavity, or the duct of the human body.

In general, endoscopes are relatively complex electromechanical appliances that present relatively high fabrication costs. Given their price, endoscopes are reused several times after being cleaned or decontaminated between uses.

Nevertheless, for several years, given the reduction in the cost of electronic components, it has been found possible to make a non-reusable separable medical instrument that is connected to a reusable connection support. Furthermore, making a non-reusable medical instrument, i.e. an instrument for single use, makes it possible to use materials that do not need to withstand cleaning, sterilization, or decontamination. Furthermore, insofar as lifetime is no longer a major criterion, such a medical instrument presents a reduced fabrication cost.

Nevertheless, a problem associated with using a single-use medical instrument relates to the possibility of the medical instrument being used on another patient. The reuse of such a medical instrument represents a danger even if such an instrument has been subjected to a decontamination or cleaning procedure, insofar as such an instrument is not designed to be subjected to such treatment.

In an attempt to counter multiple use of a single-use medical instrument, patent application WO 2010/066187 describes an endoscope in which the separable medical instrument includes a camera and a non-volatile memory. Such an endoscope has a system for detecting connection between the separable medical instrument and the support that enables operation of the camera to be controlled. The non-volatile memory records the utilization time of the camera. A control unit stops operation of the camera when the utilization time stored in the non-volatile memory is reached.

In practice, it is found that blocking use of the endoscope at the end of a predetermined duration is very inflexible. Under certain circumstances, the inspection procedure can encounter difficulties and the procedure cannot be finished during the allotted utilization time. Furthermore, the practitioner may find it necessary on a given patient to renew the inspection procedure, and that would impose using another endoscope.

Patent application EP 1 935 327 describes an imaging system having an endoscope that is separable from a connection support. That endoscope is provided with a memory containing endoscope identity data. The imaging system also has an imaging appliance for processing the images taken by the endoscope. The imaging system also has a system for detecting connection between the endoscope and the connection support. Such a system seeks to make it possible to identify the endoscope and to update parameters for proper operation of the endoscope.

On the same lines, Document US 2004/1064019 describes an imaging system having an endoscope that is separable from a connection support. The imaging system has a unit enabling the light source of the endoscope to be controlled.

Such documents do not provide a solution to the problem of controlling multiple use of a single-use medical instrument.

Patent application EP 1 679 030 describes an endoscope that is separable from a support. That document describes means for updating parameters of the imaging system.

Once more, such a document does not provide a solution to the problem of controlling safety in use of a single-use medical instrument.

Document EP 1 864 607 describes a system for managing the cleaning and disinfection of an insertion tube of an endoscope. For that purpose, the management system includes an acquisition unit adapted to acquire information about an endoscopic examination coming to an end in order to be capable of configuring the cleaning and disinfection device accordingly. Such a system is designed to manage the cleaning of an endoscope, but it is not adapted to control the use of a single-use endoscope.

The present invention seeks to remedy the drawbacks of the prior art by proposing an imaging system comprising a separable medical instrument of use that is to be controlled in order to avoid any contamination of patients, while nevertheless presenting flexibility in use.

Another object of the invention is to propose an imaging system in which use of the separable medical instrument is controlled, while not limiting the length of time it can be used and while allowing it to be reused under the responsibility of the user and while maintaining traceability of operation.

In order to achieve such an object, the imaging system comprises:
a medical instrument that is separable from a connection support, the separable medical instrument including an insertion tube provided with a non-volatile memory and a viewing system forming a portion of an imaging appliance;
an imaging appliance including a processor and image display unit connected to the viewing system; and
a control unit enabling the operation of the imaging appliance to be controlled and connected to a system for detecting connection between the medical instrument and the connection support.

According to the invention, the non-volatile memory contains identity data of the medical instrument associated with data concerning utilization of the imaging appliance and including at least one flag concerning previous use of the medical instrument, this utilization data being sent by the control unit on each connection, and the control unit allows the imaging appliance to operate only if the utilization data of the imaging appliance is supplied by a user with the help of an interface connected to the control unit, the control unit on each connection acting to read the identity data of the medical instrument and the data concerning utilization of the imaging appliance as stored in the non-volatile memory.

In addition, the system of the invention may also present in combination at least one and/or more of the following additional characteristics:
the control unit allows the imaging appliance to operate only if the user uses the interface to supply as utilization data an identity that is authorized by said control unit;
the control unit requires the user to use the interface to supply data associated with the patient as utilization data;
the control unit acts on each connection to record in the non-volatile memory the identity of the user as data concerning utilization of the imaging appliance;
the control unit acts on each connection to store in the non-volatile memory data associated with the patient as data concerning utilization of the imaging appliance;
the control unit acts on each connection to store in the non-volatile memory the date and the time of the beginning and of the end of the connection as data concerning utilization of the imaging appliance;
the control unit stores a flag concerning previous use of the medical instrument in the non-volatile memory as data concerning utilization of the imaging appliance;
the control unit acts via a display screen or the interface to require authorization from the user for any new use of a medical instrument for which the flag indicates there has been previous use of said medical instrument; and
the control unit acts on each connection to store the utilization data of the imaging appliance in a storage unit.

Various other characteristics appear from the following description made with reference to the accompanying drawings that show embodiments of the invention as non-limiting examples.

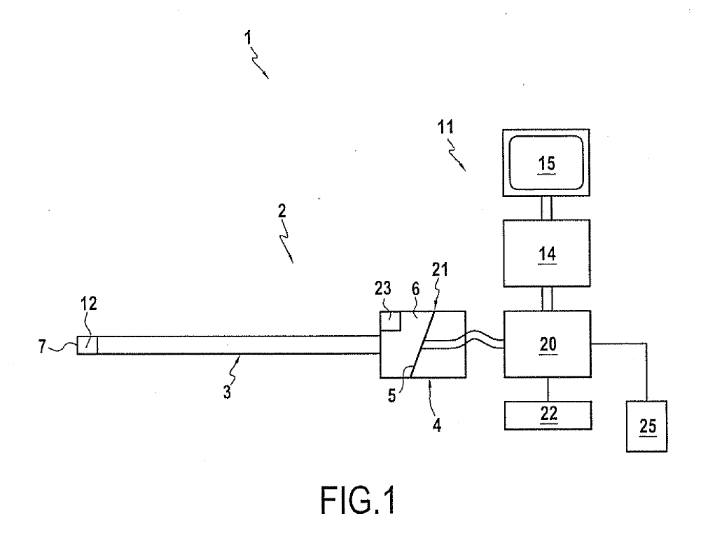

FIG. 1 is a diagrammatic view showing an imaging system in accordance with the invention.

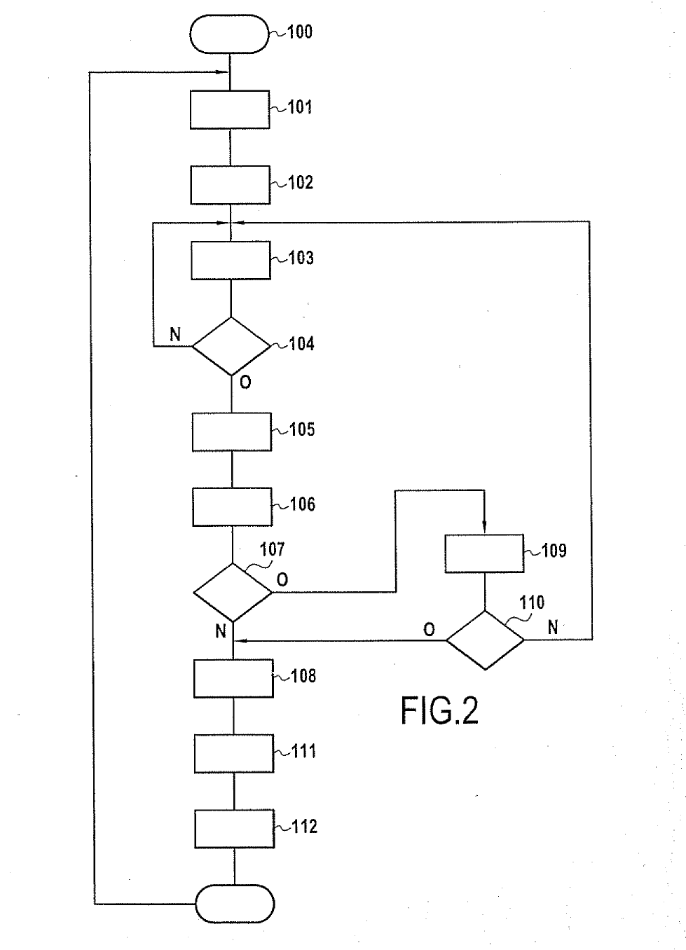

FIG. 2 shows an example of an algorithm for explaining the principle on which the invention operates.

As can be seen more clearly in FIG. 1, the invention relates to a medical imaging system 1 comprising an endoscope 2 suitable for accessing and inspecting any portion of the human body from a natural or an artificial access passageway. In conventional manner, the endoscope 2 makes it possible to inspect tissues on the surface or in-depth, a hollow organ, a cavity, or any duct in the human body for therapeutic, surgical, or diagnostic purposes. The invention relates to various types of endoscope that are adapted to organs that are accessible via a natural passageway such as a laryngoscope, a bronchoscope, an esophagoscope, a gastroscope, a duodenoscope, a cystoscope, a hystereoscope, and a coloscope, for example. The invention also relates to any type of endoscope that also makes it possible to access via passageways made artificially in the human body.

In conventional manner, the endoscope 2 comprises a medical instrument 3 that is separable from a connection block or support 4. The connection block or support 4 is generally in the form of a handle or of a robot arm. The medical instrument 3 is temporarily mounted relative to the connection support 4. Between the separable instrument 3 and the connection support 4, the endoscope 2 thus includes a connection and disconnection system 5 adapted to establish mechanical and electrical connection rapidly while also providing the advantage of enabling the medical instrument 3 to be separated easily from the connection support 4. The connection and disconnection system 5 is not described insofar as it is well known to the person skilled in the art and does not specifically form part of the invention.

In general, the medical instrument 3 is in the form of an insertion tube presenting greater or lesser flexibility and made of a material that is biocompatible. The insertion tube possesses a proximal portion 6 for connection to the connection support 4, and at its opposite end it possesses a distal portion 7 forming the head of the medical instrument 3. In a variant embodiment, the endoscope 2 includes a device enabling the distal portion 7 of the instrument 3 to be folded or deflected. Naturally, it should be observed that the insertion tube of the medical instrument 3 may be rigid, such that the endoscope is not fitted with a deflecting system for its distal portion.

The imaging system 1 of the invention also includes an imaging appliance 11 including a viewing system 12 fitted to the medical instrument 3. The viewing system 12 is an imaging system such as a camera mounted at the distal end 7 of the medical instrument 3 in order to observe or inspect an internal portion of the human body. For example, such a viewing system 12 comprises an image forming system connected by a transmission bundle that extends inside the medical instrument 3 to an image processor unit 14 connected to an image display screen 15. The imaging appliance 11 is not described in greater detail insofar as it is well known to the person skilled in the art and does not specifically form part of the invention.

The imaging system 1 also has a control unit 20 enabling the operation of the imaging appliance 11 to be controlled. This control unit 20 is connected to a system 21 for detecting connection between the medical instrument 3 and the connection support 4. The control unit 20 also has a man/machine interface 22 enabling a user to input data or to operate the control unit 20. This man/machine interface may be a keypad, a mouse, a smart card reader, or a touch-sensitive screen, for example, or the display screen 15 of the imaging appliance 1.

According to a characteristic of the invention, the medical instrument 3 is provided with at least one non-volatile memory 23. This non-volatile memory, such as an electrically erasable programmable read only memory (EEPROM) is connected to the control unit 20 to enable data to be transferred between the control unit and the non-volatile memory 23. Such a non-volatile memory 23 contains data for identifying the medical instrument 3 with which it is associated. For example, the non-volatile memory 23 contains as data for identifying the associated medical instrument: a serial number; references of the manufacturer; a use-by date; . . . . Furthermore, as explained below in the description, the non-volatile memory 23 also contains data associated with the use of the imaging appliance 11.

The control unit 20 makes it possible to control the operation of the imaging appliance 11. For this purpose, the control unit 20 enables the imaging appliance 11 to operate only if the user of the endoscope 2 has been identified with the control unit 20. Access to the imaging appliance 11 is authorized if any only if the user gives the control unit 20 identification that is recognized and authorized by the control unit 20. By using the interface 22, the user thus provides the control unit 20 with identity data considered as being utilization data in the description below. Such utilization data is associated with each authorized user, and may be biometric data or a confidential access code. Naturally, the control unit 20 contains prerecorded data relating to users in order to authorize or refuse any access requested to the imaging appliance 11.

From the above, it follows that the control unit 20 records as utilization data in the non-volatile memory 23 at least the identity of the user. In a preferred variant embodiment, the control unit 20 also records as utilization data in the non-volatile memory 23 data that is associated with the patient, such as for example the identity of the patient, etc. As explained in the description below, on each connection made between the support 4 and the medical instrument 3, the control unit 20 also records in the non-volatile memory 23 as data concerning the utilization of the imaging appliance 11 the date and the time of the beginning and of the end of the connection of the medical instrument 3 with the connection support 4.

The control unit 20 also records in the non-volatile memory 23 as data concerning the utilization of the imaging appliance 11, a flag concerning previous use of the medical instrument 3. Thus, each non-volatile memory 23 in the medical instrument 3 contains a flag making it possible to know whether the medical instrument 3 has or has not already been used with the imaging appliance 11. When the medical instrument 3 is new, the flag stored in the associated non-volatile memory 23 does not indicate any previous use. When the medical instrument 3 has already been used, the control unit 20 modifies the state of the flag recorded in the associated non-volatile memory 23, such that this flag indicates that the medical instrument 3 has already been used previously.

As explained in the description below, an already-used medical instrument 3 may be used subsequently. Under such circumstances, the control unit 20 records this information in its non-volatile memory 23, such that the flag indicates a second use of the medical instrument 3.

In a preferred embodiment, the control unit 20 acts on each connection of a medical instrument 3 to record the utilization data of the imaging appliance 11 in a storage unit or database 25 connected to the control unit.

On each connection between the medical instrument 3 and the connection support 4, the control unit 20 also reads all or some of the identity data of the medical instrument 3 and the utilization data of the imaging appliance 11 stored in the non-volatile memory 23. Thus, on each connection of a medical instrument 3 to a connection support 4, the user is in a position to know the history of the medical instrument 3 and to decide whether or not to use such a medical instrument 3.

In another variant embodiment, on first connection between the support 4 and the medical instrument, the control unit 20 also records in the non-volatile memory 23, as data concerning utilization of the imaging appliance 11, the identifier of the imaging appliance 11 to which the medical instrument 3 is connected. Thus, each imaging appliance 11 is given a different identifier, such as a serial number.

When connecting a medical instrument 3 that has already been the subject of previous use, the control unit 20 allows the imaging appliance 11 to operate only if the identifier of the imaging appliance recorded in the non-volatile memory 23 corresponds to the identifier of the imaging appliance to which said medical instrument 3 is connected. In other words, if a medical instrument 3 that has previously been used with one imaging appliance, is subsequently connected to another imaging appliance, then the control unit 20 of that other imaging appliance prevents it from being operated. In this preferred embodiment, a medical instrument 3 can thus be used with only one imaging appliance 11.

The imaging system 1 of the invention thus allows the medical instrument 3 to be reused under the entire responsibility of the user (practitioner). The practitioner is in a position to decide, for whatever reason, whether to repeat or restart an examination by confirming the re-utilization request while being identified with the control unit 20.

The use of a non-volatile memory 23 as means for verifying single use makes it possible to be fully aware of the state of the endoscope and of the examination in normal operation mode:
medical instrument 3 not yet used, examination not yet begun;
medical instrument 3 being used, examination begun; and
medical instrument 3 used, examination finished.

The imaging system 1 of the invention also makes it possible to identify abnormal modes of operation on the endoscope being switched on:
medical instrument 3 used but examination still in progress (accidental disconnection of the medical instrument 3 or accidental switching off of the imaging appliance). The practitioner must confirm reuse of the endoscope; and
medical instrument 3 used, examination finished. The practitioner is repeating an examination and must validate reuse of the endoscope.

The fact of using a control unit 20 makes it possible to have redundant data concerning the data contained in the medical instrument 3. The information concerning all of the medical instruments 3 used is stored in the database 25. The information stored in the database 25 also contains the confirmations of departures from normal operation as given by the practitioner and the identity of the practitioner.

The principle of operation of such an imaging system for validating single use of the consumable has two strong points:
flexibility in use, since such a system does not forbid use nor does it block use after a given utilization time;
safety, by performing two checks or a cross-check against involuntary reuse of the endoscope, with this being achieved by means of the non-volatile memory of the medical instrument 3 and by means of the control unit 20.

The imaging system of the invention thus makes it possible to make safe single use of a medical instrument for a single patient under the responsibility of the user (practitioner).

FIG. 2 is a diagram for illustrating the operation of the imaging appliance of the invention.

After a step 100 of starting and initializing the control unit 20, the practitioner is invited to provide his or her own identity together optionally with the identity of the patient (step 101). It should be observed that it is required to input the practitioner's identity in order to be able to continue with the procedure for using the endoscope. As explained above, the user provides identity data in order to enable the request for utilization to be authorized. Naturally, any type of procedure for verifying the identities of practitioners may be implemented (confidential code, biometric data, etc.).

Starting from a request to begin an examination (step 102), there is a step 103 for detecting connection between the medical instrument 3 and the connection support 4. This detection step is performed by the system 21. This step is repeated so long as the medical instrument 3 is not connected to the connection support 4 (step 104). Once the medical instrument 3 is connected to the connection support 4, the control unit 20 acts during a step 105 to read the data contained in the non-volatile memory 23. This data corresponds to the identity data of the medical instrument associated with utilization data of the imaging appliance 11, indicating that the medical instrument 3 has not previously been used, or on the contrary that it has previously been used. This reading is followed by a step 106 of recording data in the database 25.

During the following step 107, a test is performed on the flag concerning previous use of the medical instrument 3. If the flag indicates a new and not-yet used state, then the control unit 20 moves onto the following step 108 of storing utilization data of the imaging appliance 11, and in particular of storing it in the non-volatile memory 23. If the flag indicates there has been previous use of the medical instrument 3, then the control unit 20 uses the display screen 15 or the interface 22 to warn the user that it is possible to force use of the medical instrument 3 (step 109).

If the flag indicates that there has been previous use of the medical instrument 3, then the control unit 20 reads the identifier of the imaging appliance stored in the non-volatile memory 23. In the event that the identifier recorded in the non-volatile memory 23 of the medical instrument 3 corresponds to the identifier of the imaging appliance to which the medical instrument is connected, then the control unit 20 authorizes this new use of the medical instrument 3. In contrast, in the event that the identifier recorded in the non-volatile memory 23 of the medical instrument 3 does not correspond to the identifier of the imaging appliance to which it is connected, then the control unit 20 prevents operation of the imaging appliance 11 and warns the user.

If the user in test step 110 decides not to use the medical instrument 3, then the method consists in returning to step 103 of detecting connection between a medical instrument 3 and the connection support 4.

If the user decides to force use of this already-used medical instrument 3, then the control unit 20 acts in the following storage step 108 to store this new use in the database 25 in order to provide traceability.

The storage step 108 also consists in storing the utilization data of the imaging appliance 11 in the non-volatile memory 23, i.e. the identity data of the user, the data associated with the patient, the date and time of the beginning of the connection, the flag indicating use of the medical instrument 3, and the identifier of the imaging appliance 11.

Thereafter, the examination procedure using the endoscope 2 can take place. During examination (step 111), the control unit 20 periodically updates the date and the time so that this information is recorded in the non-volatile memory 23. During the end-of-examination step 112, the date and time of the end of use are recorded both in the non-volatile memory 23 and in the database 25.

If at the end of an examination using the endoscope 2, the medical instrument 3 of the endoscope is once more connected to the connection support 4, then the control unit 20 acting in step 105 to read the content of the non-volatile memory 23 attached to the medical instrument 3 serves to determine whether such a medical instrument 3 has already been the subject of previous use. Such a medical instrument 3 can be reused by a user (step 110) under the user's entire responsibility, unlike known prior solutions.

The invention is not limited to the examples described and shown since various modifications can be made thereto without going beyond the ambit of the invention.

1. An imaging system comprising:
a medical instrument (3) that is separable from a connection support (4), the separable medical instrument including an insertion tube provided with a non-volatile memory (23) and a viewing system (12) forming a portion of an imaging appliance;
an imaging appliance (11) including a processor and image display unit connected to the viewing system; and
a control unit (20) enabling the operation of the imaging appliance (11) to be controlled and connected to a system (21) for detecting connection between the medical instrument (3) and the connection support (4);
the system being characterized in that the non-volatile memory (23) contains identity data of the medical instrument (3) associated with data concerning utilization of the imaging appliance and including at least one flag concerning previous use of the medical instrument (3), this utilization data being sent by the control unit (20) on each connection, and in that the control unit (20) allows the imaging appliance (11) to operate only if the utilization data of the imaging appliance is supplied by a user with the help of an interface (22) connected to the control unit (20), the control unit (20) on each connection acting to read the identity data of the medical instrument (3) and the data concerning utilization of the imaging appliance (11) as stored in the non-volatile memory (23).

2. An imaging system according to claim 1, characterized in that the control unit (20) allows the imaging appliance (11) to operate only if the user uses the interface (22) to supply as utilization data an identity that is authorized by said control unit.

3. An imaging system according to claim 2, characterized in that the control unit (20) requires the user to use the interface (22) to supply data associated with the patient as utilization data.

4. An imaging system according to claim 1 characterized in that the control unit (20) acts on each connection to record in the non-volatile memory (23) the identity of the user as data concerning utilization of the imaging appliance.

5. An imaging system according to claim 1, characterized in that the control unit (20) acts on each connection to store in the non-volatile memory (23) data associated with the patient as data concerning utilization of the imaging appliance.

6. An imaging system according to claim 4, characterized in that the control unit (20) acts on each connection to store in the non-volatile memory (23) the date and the time of the beginning and of the end of the connection as data concerning utilization of the imaging appliance.

7. An imaging system according to claim 1, characterized in that the control unit (20) stores a flag concerning previous use of the medical instrument (3) in the non-volatile memory (23) as data concerning utilization of the imaging appliance.

8. An imaging system according to claim 1, characterized in that the control unit (20) acts during connection of the medical instrument (3) with the connection support (4) to store an identifier of the imaging appliance in the non-volatile memory (23) as data concerning utilization of the imaging appliance, and in that the control unit (20) authorizes operation of the imaging appliance (11) only if the identifier of the imaging appliance as recorded in the non-volatile memory (23) corresponds to the identifier of the imaging appliance to which the medical instrument (3) is connected.

9. An imaging system according to claim 8, characterized in that the control unit (20) acts via a display screen (15) or the interface (22) to require authorization from the user for any new use of a medical instrument (3) for which the flag indicates there has been previous use of said medical instrument (3).

10. An imaging system according to claim 1, characterized in that the control unit (20) acts on each connection to store the utilization data of the imaging appliance (11) in a storage unit (25).
